(19) United States
(12) Patent Application Publication
(10) Pub. No.: US 2025/0025137 A1
(43) Pub. Date: Jan. 23, 2025

(54) DEVICE WITH DISCRETE HYDROPHILIC POROUS COMPONENTS AND METHOD OF MAKING THE SAME

(71) Applicant: Porex Corporation, Fairburn, GA (US)

(72) Inventor: Michael Gerald Arthur, Douglasville, GA (US)

(21) Appl. No.: 18/577,136

(22) PCT Filed: Jul. 8, 2022
(86) PCT No.: PCT/US2022/036450

§ 371 Date: Jan. 5, 2024

Related U.S. Application Data
(60) Provisional application No. 63/220,180, filed on Jul. 9, 2021.

(51) Int. Cl. A61B 10/00; B01L 3/00

(52) U.S. Cl. CPC A61B 10/0096; B01L 3/502707; B01L 2300/0829

(57) ABSTRACT

A device (10) for storing or delivering an aqueous solution and methods for manufacturing the device. The includes a non-porous hydrophobic substrate (100); and a plurality of hydrophilic porous protrusions (102) discretely distributed on the non-porous hydrophobic substrate and integrally formed on the non-porous hydrophobic substrate. Each of the plurality of hydrophilic porous protrusions is configured to absorb and hold an aqueous solution e.g., an ink, a biological liquid sample, or paint. The hydrophilic porous protrusions can have same or different shapes. The protrusions can be distributed in any desired pattern.

### CROSS-REFERENCES TO RELATED APPLICATIONS

This application claims the benefit of U.S. Patent Application Ser. No. 63/220,180, filed on Jul. 9, 2021, the entirety of which is incorporated by reference herein.

### BACKGROUND

Porous materials, including metal, ceramic, glass and polymeric materials, have increasingly been used in a variety of applications, such as filtration, aeration, wicking, implant and other biomedical devices. For example, porous polymeric materials can be used in medical devices that serve as substitute blood vessels, synthetic and intra-ocular lenses, electrodes, catheters, and extra-corporeal devices such as those that are connected to the body to assist in surgery or dialysis. Porous polymeric materials can also be used as filters for the separation of blood into component blood cells and plasma, microfilters for removal of microorganisms from blood, and coatings for ophthalmic lenses to prevent endothelial damage upon implantation. Porous materials have also been used in diagnostic devices such as lateral flow devices, flow through devices and other immunoassay devices.

The hydrophobic nature of many polymers, however, has limited the usefulness of porous materials made from them. Therefore, attempts have been made to modify the surface properties of the porous materials. Various methods were contemplated to achieve such modifications on the surface properties.

A device with sintered porous plastic spots for receiving liquid and delivery of the liquid has been disclosed before in Applicant's U.S. Pat. No. 9,101,311. The '311 patent discloses cards for sample storage and delivery using the sintered porous plastic. The sintered porous plastic could be hydrophilic disks disposed in cavities or holes in the card for liquid sample storage or delivery. In such card devices, the hydrophilic disks may pop out from the cavities in the card. Another device comprising a discrete hydrophilic-hydrophobic porous material is described in U.S. patent publication 2003/0134100. The device is a single piece of porous media with discrete hydrophilic regions surrounded by hydrophobic boundaries.

### BRIEF SUMMARY

According to some embodiments, a device including a substrate with multiple porous protrusions for storing and/or delivering aqueous solution is described. Specific embodiments relate to devices with specific configuration, shapes and sizes of the substrate and multiple porous protrusions. The device can be advantageously used for various applications such as transferring samples, ink, creating patterns, etc. and accordingly different configurations of multiple porous protrusions are possible.

According to an aspect of the present disclosure, a device for storing or delivering an aqueous solution. The device includes a non-porous hydrophobic substrate, and a plurality of hydrophilic porous protrusions. The plurality of hydrophilic porous protrusions are discretely distributed on the non-porous hydrophobic substrate and integrally formed on the non-porous hydrophobic substrate. Each of the plurality of hydrophilic porous protrusions is configured to absorb and hold an aqueous solution.

In some embodiments, the non-porous hydrophobic substrate is made of solid non-porous polymeric material sheet having a first melting temperature. The non-porous hydrophobic substrate is made of high density polyethylene (HDPE) polypropylene, low density polyethylene (LDPE), ethylene vinyl acetate (EVA), polystyrene, polyesters, PMMA, polycarbonate or nylons, or any combination thereof.

In some embodiments, the plurality of hydrophilic porous protrusions are made polymeric powders having a second melting temperature. The second melting temperature is higher than the first melting temperature of the non-porous hydrophobic substrate. The hydrophilic porous protrusions are made of high density polyethylene (HDPE), ultrahigh molecular polyethylene. Each of the plurality of hydrophilic porous protrusions has a height (H) and the height of each hydrophilic porous protrusions is the same, such that tops of each hydrophilic porous protrusions define an upper plane raised a distance H from the non-porous hydrophobic substrate. The plurality of hydrophilic porous protrusions includes one or more protrusions of different heights from the non-porous hydrophobic substrate. A shape of each of the plurality of hydrophilic porous protrusions includes at least one of a cylinder, a polygonal prism, a cone, a torus, a star, a half-sphere, a rectangle, a square, or a pyramid shapes. A subset of the plurality of hydrophilic porous protrusions forms a pattern, a picture, or a geographic shape on the non-porous hydrophobic substrate. The pattern includes the plurality of hydrophilic porous protrusions evenly distributed in n rows and n columns. The plurality of hydrophilic porous protrusions includes one or more protrusions having same size and/or shape. In some embodiments, one or more protrusions can have different sizes and/or shapes.

In some embodiments, each of the plurality of hydrophilic porous protrusions is configured to uptake aqueous solution from 0.1 μl to 1 ml, from 0.2 μl to 500 μl, from 0.3 μl to 200 μl, from 0.5 μl to 100 μl, from 1 μl to 50 μl, or from 1 μl to 20 μl. The aqueous solution includes at least one of an ink, a biological solution or sample, a beverage, or an environment solution. In some embodiments, the non-porous hydrophobic substrate is transparent to allow visibility of colors or changes in color of the aqueous solution.

According to another aspect of the present disclosure, a method of manufacturing a device for storing or delivering a liquid sample is described. The method includes filling multiple discretely distributed cavities of a mold with a first polymeric material, placing a polymeric sheet of a second material over the cavities, heating the mold above a melting temperature of the first material, cooling the first polymeric material in the cavities and the polymeric sheet so that the first polymeric material is integrally attached to the polymeric sheet. The second material has a melting temperature lower than a melting temperature of the first material.

In some embodiments, the first polymeric material includes a powder and filling of the mold involves compactly packing the powder of the first material in the cavities of the mold to form hydrophilic porous protrusions. The heating of the mold involves heating the mold from a first side in a temperature range from 300° C. to 360° C.

### BRIEF DESCRIPTION OF THE DRAWINGS

Various embodiments in accordance with the present disclosure will be described with reference to the drawings, in which:

FIG. 1A is a top view and FIG. 1B is a side view of a first device configured with a first pattern (e.g., a matrix pattern) of porous protrusions on a non-porous substrate, according to some embodiments of the present disclosure.

FIG. 2 a second device configured with porous protrusions in a second alternate exemplary pattern distributed on a non-porous substrate, according to some embodiments of the present disclosure.

FIG. 3 a third device configured with porous protrusions in a third alternate exemplary pattern distributed on a non-porous substrate, according to some embodiments of the present disclosure.

FIG. 4 a method of manufacturing the devices of FIGS. 1-3, according to some embodiments of the present disclosure.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

The present disclosure relates to a device with discrete hydrophilic porous components and method of making and using the device. The discrete hydrophilic porous components may be integrally formed with a base structure/substrate that can be made of a solid (or otherwise nonporous) substrate media. The hydrophilic porous components can receive and wick any type of fluid, such as an ink, a biological liquid sample, paint, or any other appropriate fluid.

More specifically, the device may include a non-porous/solid substrate with two sides. A first side has the discrete hydrophilic porous components fused thereto. Fusing the components can be a more cost-effective solution compared to providing removable porous components. The second side provides a generally flat surface so that the device may be positioned on a table for working purposes.

The device could receive the liquids from many types of devices, such as printers, writing instruments, brushes, pipettes, and other liquid handling devices.

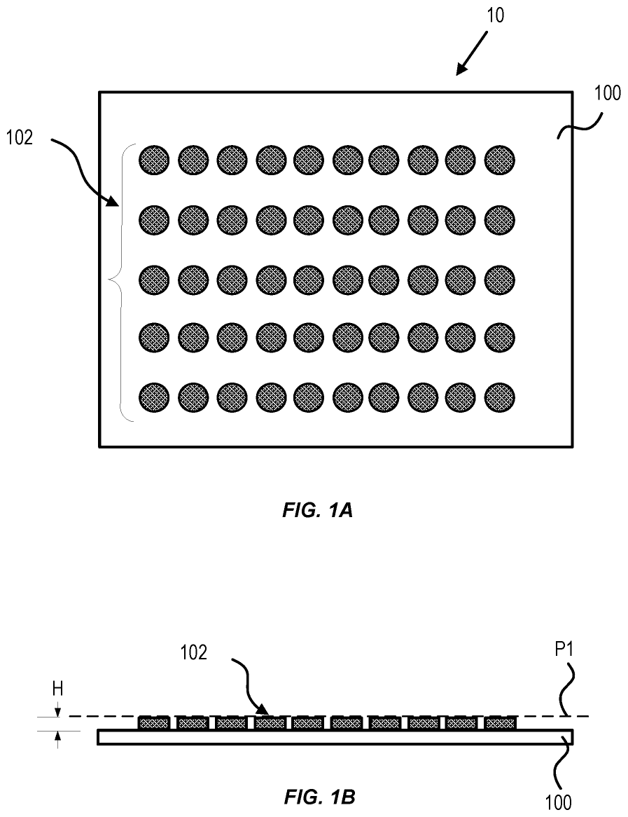

The discrete hydrophilic porous components may be disposed in an array across the first side. The array may be a symmetric pattern of lines, for example, as shown in FIG. 1. However, the array may be randomly-aligned or provided in any other desired pattern. The outline of the base structure/substrate may be rectangular, square, triangular, circular, pentagonal, octagonal, or any other appropriate shape. The shape of the discrete hydrophilic porous components similarly may be rectangular, square, triangular, circular, pentagonal, octagonal, or any other appropriate shape as described further below.

Examples of devices with components for receiving liquid and delivery of the liquid has been disclosed in U.S. Pat. No. 9,101,311, which is incorporated herein by reference in its entirety. The '311 patent discloses cards for sample storage and delivery comprising sintered porous plastic. The sintered porous plastic could be hydrophilic disks in the card for liquid sample storage. In this invention, the porous disks are in the cavities or holes in the cards. As another example, U.S. patent application no. 2003/0134100, which is incorporated herein by reference in its entirety, discloses a porous material and the method of making. The '100 patent application describes a single piece of porous media with hydrophobic and hydrophilic regions.

In the present disclosure, one example of a nonporous base substrate with discrete hydrophilic porous components fused thereto is illustrated by FIGS. 1-3. As shown, the device may include a flat non-porous plastic substrate with two sides. On one side of the non-porous plastic substrate there may be provided an array of hydrophilic porous components extending up therefrom. The height of the components may be anywhere from a millimeter or two up to an inch or two. The height may be dependent upon an intended use of the device. For example, if the device is intended to receive and store biological samples, more surface area may be needed. Alternatively, if the device is intended to receive a colorant or other aesthetic design function, less surface area for the components may be needed and they may only extend only up a short distance from the base. The height of hydrophilic porous component could be the same or difference.

The interior shapes of the hydrophilic porous components may be circles or squares, but it should be understood that alternate shapes are possible, and may be varying sizes. The flat sheet/substrate may be a non-porous solid polymeric material. In many embodiments, the raised shapes may only be positioned on one side of non-porous solid sheet.

The hydrophilic porous components may be sintered porous polymeric materials. In a preferred embodiment, the hydrophilic porous components are sintered porous plastic, or sintered porous elastic materials. The sintered hydrophilic porous components may be the sintered porous media described in U.S. Pat. Nos. 8,141,717 and/or 9,101,311, both of which are incorporated herein by reference in its entirety. In a specific embodiment, the hydrophilic porous component could be sintered polyethylene.

The hydrophilic porous components may have any physical shapes from the top view, such as circles, triangles, squares, and other polygons, ellipsoids, parallelograms, or combination of them. The hydrophilic porous components may also have 3D shape of cylinder, polygonal prism, cone, torus, star, half-sphere, pyramid shapes. The hydrophilic porous components may form patterns, pictures, or geographic shapes on the non-porous solid substrate. Each hydrophilic porous component may absorb and hold aqueous solutions by capillary force. The aqueous solution could be inks, biological solutions, beverages, environment solutions, etc.

The hydrophilic porous component may have the same or different size, each hydrophilic porous component could uptake aqueous solution from 0.1 μI to 1 ml, from 0.2 μI to 500 μI, from 0.3 μI to 200 μI, from 0.5 μI to 100 μI, from 1 μI to 50 μI, or from 1 μI to 20 μI. The hydrophilic porous components can be white and opaque.

The aqueous solution or components in the aqueous stored in the hydrophilic porous components may be transferred to another solid surface, or container by capillary force or extraction with another liquid. In other examples, the aqueous solution applied to the hydrophilic porous components may be for artistic or aesthetic purposes. For example, ink or paint may be applied to the hydrophilic porous components to provide an interesting design or for the device to function as a drawing surface, by filling in each hydrophilic porous component with a different color. If designed as such, the ink or paint may be rinsed out of the hydrophilic porous components for another use of the device.

The non-porous solid substrate may be polyethylene, polypropylene, EVA, polystyrene, polyesters, PMMA, polycarbonate or nylons, or any other appropriate material or combination thereof. In a specific embodiment, the substrate is polyethylene. The non-porous solid substrate may be any color or any combination of colors. The non-porous solid substrate could be opaque or transparent.

The device may be modified, reused and regenerate using tradition washing process. Such as rinsing or dish washing.

### Method of Making

The device in present disclosure may be made by a molding process. A mold (e.g., a metallic mold or silicone mold) with multiple cavities may be filled with polymeric powders. In one example, the polymeric powders are high molecular weight polyethylene or high density polyethylene (HDPE). The cavities have a shape of a desired shape for the hydrophilic porous components. A polymeric sheet may then be placed on top or over the filled cavities. In one example, the polymeric sheet is low density polyethylene (LDPE). The mold may then be heated above the melting temperature of the polymeric powder for an appropriate period of the time and then cooled to the room temperature. The device can be removed from the mold by lifting the polymeric sheet. It is generally desirable that the heating temperature does not melt or otherwise cause substantial deformation of the polymeric sheet so that it functions as a base substrate. The result of this process can be that the polymeric powders become sintered into porous components and be fused on the surface of the polymeric sheet. Exemplary sintering conditions and processes are described in U.S. Pat. Nos. 8,141,717; 9,101,311; and 9,696,241, all of which are incorporated herein by reference in its entirety.

### Application of the Device

The device in present disclosure may be used in any application for absorbing aqueous solution on the discrete hydrophilic porous components. The absorbed aqueous solution and composition may be further transferred to target object surfaces. The target objects can be papers, glasses, plastics, or metals. The aqueous solution or components in the aqueous solution stored in the hydrophilic porous components may be transferred to another solid surface, or container by capillary force or extraction with another liquid.

The device in present disclosure may be used as a toy, such as, stamp or stencil or drawing surface. The device may also be used as a liquid sampling and storage device for biological or environment solutions. Accordingly, the device can function as an intermediate holding device for ultimate transfer of the aqueous solution elsewhere.

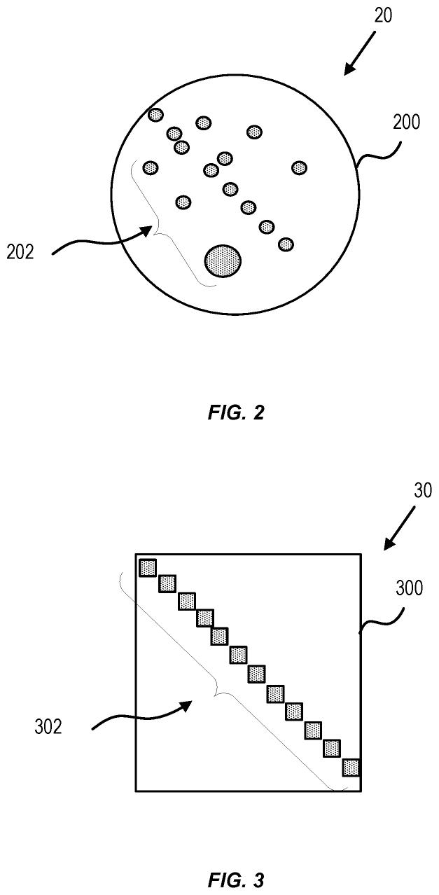

FIGS. 1-3 illustrate example devices for storing and/or delivering an aqueous solution. The devices 10, 20, and/or 30 have different substrate shapes and porous protrusions. Referring to FIG. 1, the device 10 may include a non-porous hydrophobic substrate 100; and a plurality of hydrophilic porous protrusions 102. The plurality of hydrophilic porous protrusions 102 may be discretely distributed on the non-porous hydrophobic substrate 100 and integrally formed on the non-porous hydrophobic substrate 100. Each of the plurality of hydrophilic porous protrusions 102 may be configured to absorb and hold an aqueous solution (e.g., by capillary forces). Each hydrophilic porous protrusion is spaced from and fluidically isolated from other hydrophilic porous protrusions and separated by the hydrophobic substrate so that the aqueous solution from one porous protrusion does not enter the other porous protrusion. In an example, the hydrophobic substrate 100 (or a substrate 300 in FIG. 3) can be rectangular or square shape. In an example, as shown in FIG. 2, the hydrophobic substrate 200 can be circular in shape. It should be understood, however, that any appropriate shape for the hydrophobic substrate is possible and considered within the scope of this disclosure.

The plurality of hydrophilic porous protrusions 102 may include one or more protrusions having same size and/or shape. Shapes of the plurality of hydrophilic porous protrusions 102 may be at least one of a cylinder, a polygonal prism, a cone, a torus, a star, a half-sphere, a rectangle, a square, or a pyramid shapes. Further, the shapes may be arranged such that a subset of the plurality of hydrophilic porous protrusions 102 form a pattern, a picture, or a geographic shape on the non-porous hydrophobic substrate 100. The pattern may include the plurality of hydrophilic porous protrusions 102 are evenly distributed in matrix or array of n rows and n columns (e.g., 16×16 or 96×96 protrusions extending from the substrate). However, the present disclosure is not limited to array or matrix distribution and other distributions are possible. The plurality of hydrophilic porous protrusions 102 may include one or more protrusions having different sizes and/or shapes. The porosity and height of the hydrophilic porous protrusions can depend on the type of application without limiting the scope of the present disclosure. For example, the porosity can be in a range from 20% to 80% and pore sizes can range from a pore size of from about 1 μm to about 200 μm. For example, the height for storing/transferring biological sample of 2 μl may have a height of 2 mm, for storing/transferring 5 μl may have a height of 5 mm, etc.

FIGS. 2 and 3 illustrate the second and the third devices 20 and 30, respectively, having different distributions of hydrophilic porous protrusions 202 and 302, respectively. For example, in device 20, the hydrophilic porous protrusions 202 may be of different sizes and non-uniformly distributed on a circular non-porous substrate 200. In the device 30, the hydrophilic porous protrusions 302 may be of same sizes and shapes and evenly distributed along a diagonal of a square non-porous substrate 300.

Referring back to FIG. 1, the non-porous hydrophobic substrate 100 (and 200/300 in FIGS. 2-3) may be made of solid non-porous polymeric material sheet having a first melting temperature. For example, the non-porous hydrophobic substrate 100 (and 200/300) may be made of high density polyethylene (HDPE) polypropylene, low density polyethylene (LDPE), ethylene vinyl acetate (EVA), polystyrene, polyesters, PMMA, polycarbonate or nylons, or a combination thereof

The plurality of hydrophilic porous protrusions 102/202/302 may be made of polymeric powders having a second melting temperature. The second melting temperature may be higher than the first melting temperature of the non-porous hydrophobic substrate 100. For example, the hydrophilic porous protrusions 102 (and 202/302) may be made of high density polyethylene (HDPE), Ultrahigh molecular weight polyethylene. Each of the plurality of hydrophilic porous protrusions 102 may have a height (H). For example, the height can range from 1 mm to 50 mm. The height of each hydrophilic porous protrusions 102 may be the same. Tops of each hydrophilic porous protrusions 102 define an upper plane raised by a distance H from a top plane of the non-porous hydrophobic substrate 100. In some embodiments, the plurality of hydrophilic porous protrusions 102 may include one or more protrusions of different heights (e.g., a first height>a second height (H)>a third height) from the non-porous hydrophobic substrate 100.

Each of the plurality of hydrophilic porous protrusions 102 may be configured to uptake aqueous solution from 0.1 μl to 1 ml, from 0.2 μl to 500 μl, from 0.3 μl to 200 μl, from 0.5 μl to 100 μl, from 1 μl to 50 μl, or from 1 μl to 20 μl. The aqueous solution may include at least one of an ink, a dye, a biological solution or sample, a beverage, an environment solution, or any other fluid. In some embodiments, the non-porous hydrophobic substrate 100 may be transparent to allow visibility of colors or changes in color of the aqueous solution.

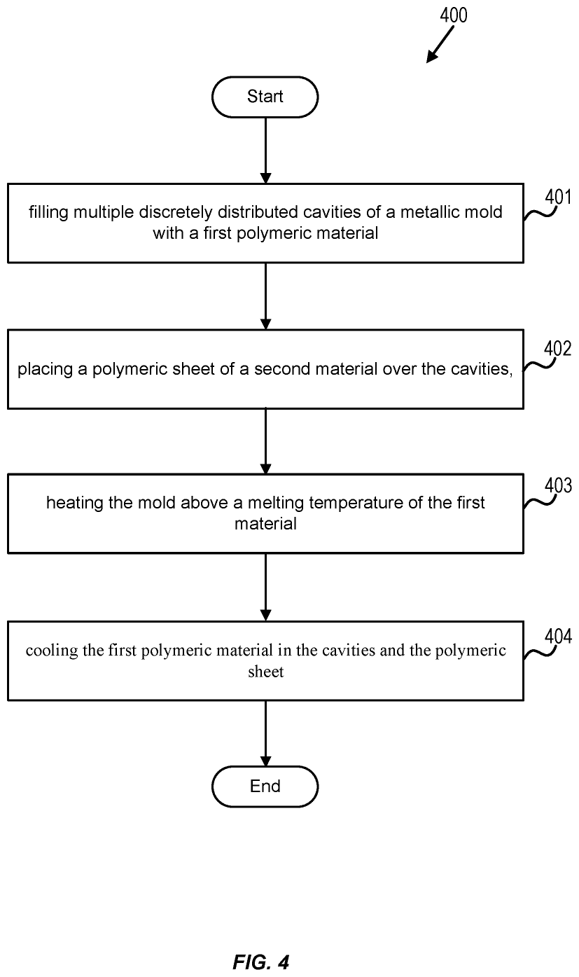

FIG. 4 illustrates a method of manufacturing a device for storing or delivering a liquid sample. In an example, a method 400 involves steps 401-404, as discussed below.

At step 401, the method involves filling multiple discretely distributed cavities of a mold with a first polymeric powder material. The first material may be high density polyethylene (HDPE), ultrahigh molecular weight polyethylene. The mold may be made of metal, silicon or other mold material. Accordingly, the filling of the cavities may involve compactly packing the powder of the first material in the cavities of the mold to form hydrophilic porous protrusions (e.g., the porous protrusions 102, 202, or 302 in FIGS. 1-3).

At step 402, the method involves placing a polymeric sheet of a second material over the cavities. The polymeric sheet forms a non-porous substrate (e.g., 100, 200, or 300 in FIGS. 1-3). The second material having a melting temperature lower than a melting temperature of the first material. The second material may include at least one of low density polyethylene (LDPE) polypropylene, ethylene vinyl acetate (EVA), polystyrene, polyesters, PMMA, polycarbonate or nylons, any other appropriate material, or any combination thereof.

At step 403, the method involves heating the mold above a melting temperature of the first material. The mold may be heated uniformly or only from a first side opposite to a second side where the polymeric sheet is placed. The heating of the mold may involve heating the mold from the first side for a specific amount of time such that the polymeric sheet melts into the cavities and the first material in the cavities fuses to the polymeric sheet. For example, the mold may be heated from 300° C. to 360° C.

At step 404, the method involves cooling the first polymeric material in the cavities and the polymeric sheet so that the first polymeric material is integrally attached or fused to the polymeric sheet. In some embodiments, the polymeric sheet may be a flat sheet or a wavy sheet that flattens out after heating.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the disclosure.

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

1. A device for storing or delivering an aqueous solution, the device comprising:
a non-porous hydrophobic substrate; and
a plurality of hydrophilic porous protrusions discretely distributed on the non-porous hydrophobic substrate and integrally formed on the non-porous hydrophobic substrate, wherein each of the plurality of hydrophilic porous protrusions is configured to absorb and hold an aqueous solution.

2. The device of claim 1, wherein the non-porous hydrophobic substrate is made of solid non-porous polymeric material sheet having a first melting temperature.

3. The device of claim 2, wherein the non-porous hydrophobic substrate is made of high density polyethylene (HDPE) polypropylene, low density polyethylene (LDPE), ethylene vinyl acetate (EVA), polystyrene, polyesters, PMMA, polycarbonate or nylons, or any combination thereof.

4. The device of claim 2, wherein, the plurality of hydrophilic porous protrusions are made polymeric powders having a second melting temperature, the second melting temperature being higher than the first melting temperature of the non-porous hydrophobic substrate.

5. The device of claim 4, wherein the hydrophilic porous protrusions are made of high density polyethylene (HDPE), ultrahigh molecular polyethylene.

6. The device of claim 1, wherein each of the plurality of hydrophilic porous protrusions comprises a height (H) and wherein the height of each hydrophilic porous protrusions is the same, such that tops of each hydrophilic porous protrusions define an upper plane raised a distance H from the non-porous hydrophobic substrate.

7. The device of claim 1, wherein the plurality of hydrophilic porous protrusions comprises one or more protrusions of different heights from the non-porous hydrophobic substrate.

8. The device of claim 1, wherein a shape of each of the plurality of hydrophilic porous protrusions comprises at least one of: a cylinder, a polygonal prism, a cone, a torus, a star, a half-sphere, a rectangle, a square, or a pyramid shapes.

9. The device of claim 1, wherein a subset of the plurality of hydrophilic porous protrusions forms a pattern, a picture, or a geographic shape on the non-porous hydrophobic substrate.

10. The device of claim 9, wherein the pattern comprises the plurality of hydrophilic porous protrusions are evenly distributed in n rows and n columns.

11. The device of claim 1, wherein the plurality of hydrophilic porous protrusions comprises one or more protrusions having same size and/or shape.

12. The device of claim 1, wherein the plurality of hydrophilic porous protrusions comprises one or more protrusions having different sizes and/or shapes.

13. The device of claim 1, wherein each of the plurality of hydrophilic porous protrusions is configured to uptake aqueous solution from 0.1 μl to 1 ml, from 0.2 μl to 500 μl, from 0.3 μl to 200 μl, from 0.5 μl to 100 μl, from 1 μl to 50 μl, or from 1 μl to 20 μl.

14. The device of claim 1, wherein the aqueous solution comprises at least one of an ink, a biological solution or sample, a beverage, or an environment solution.

15. The device of claim 1, wherein the non-porous hydrophobic substrate is transparent to allow visibility of colors or changes in color of the aqueous solution.

16. A method of manufacturing a device for storing or delivering a liquid sample, the method comprising:
filling multiple discretely distributed cavities of a mold with a first polymeric material;
placing a polymeric sheet of a second material over the cavities, the second material having a melting temperature lower than a melting temperature of the first material;
heating the mold above a melting temperature of the first material;
cooling the first polymeric material in the cavities and the polymeric sheet so that the first polymeric material is integrally attached to the polymeric sheet.

17. The method of claim 16, wherein the first polymeric material comprises a powder and wherein filling of the mold comprises:
compactly packing the powder of the first material in the cavities of the mold to form hydrophilic porous protrusions.

18. The method of claim 16, wherein heating of the mold comprises:
heating the mold from a first side in a temperature range from 300° C. to 360° C.
